(19) United States
(12) Patent Application Publication
(10) Pub. No.: US 2023/0397716 A1
(43) Pub. Date: Dec. 14, 2023

(54) COSMETIC BRUSH CLEANING DEVICE FOR CLEANING AND SANITIZING COSMETIC BRUSHES

(71) Applicant: Kimberly Lewis, Arlington, TX (US)

(72) Inventor: Kimberly Lewis, Arlington, TX (US)

(21) Appl. No.: 18/322,491

(22) Filed: May 23, 2023

Related U.S. Application Data
(60) Provisional application No. 63/345,265, filed on May 24, 2022.

(51) Int. Cl. A46B 17/06; A61L 2/10; A61L 2/24; A61L 2/26

(52) U.S. Cl. CPC A46B 17/065; A61L 2/10; A61L 2/24; A61L 2/26; A61L 2202/122; A61L 2202/14; A46B 2200/1046

(57) ABSTRACT

A cosmetic brush cleaning device for cleaning and sanitizing cosmetic brushes is disclosed. The cosmetic brush cleaning device includes a first housing, and a second housing placed over the first housing. The first housing includes a container having a first base, and first walls extending from the first base. The first walls receive brush holders. The second housing includes a second base, and second walls extending from the second base. The second base and the second walls form a brush receiving area. The first housing includes a control panel positioned adjacent to the container. The first housing includes a fan, an Ultraviolet (UV) light, and a controller. The brush holders receive cosmetic brushes. The controller operates the fan and the UV light to clean and sanitize the cosmetic brushes. The cosmetic brushes are removed from the first housing and placed in the second housing to dry the cosmetic brushes.

### CROSS-REFERENCE TO RELATED APPLICATIONS

This application claims priority to and benefit of U.S. Provisional Patent Application No. 63/345,265 filed May 24, 2022; the contents of which are incorporated in their entirety.

### FIELD OF THE INVENTION

The present invention generally relates to beauty accessories. More particularly, the present invention relates to a cosmetic brush cleaning device for cleaning and sanitizing cosmetic brushes, palettes and the like.

### DESCRIPTION OF THE RELATED ART

It is known that cosmetic or makeup brushes are often used for application of makeup or face painting. Typically, the makeup brushes have bristles made of natural or synthetic material. The bristles help to apply the makeup evenly on the skin. The makeup brushes accumulate dirt and other particles after using them for a period of time. An uncleaned makeup brush may cause infection to the skin. Further, any residue of the makeup left on the bristles weighs down the bristles. This may lead to weakening and even breaking of the bristles.

In order to overcome the problems discussed above, several devices have been proposed in the past. One such device is disclosed in a United States Publication No. 20140096801, entitled “Makeup brush cleaner and sanitizer” (“the '801 Publication”). The '801 Publication discloses an apparatus configured to hold multiple cosmetics or other makeup type brushes in specifically designed retention means such that only the brush tips/bristles of the brushes are subject to the sanitation area of the apparatus. Certain embodiments of the present apparatus provide the sanitation area of the apparatus to clean the brush tips/bristles of the retained brushes via one or more of a cleaning solution, water, ultra-violet cleaning source, ultrasonic cleaning source, gentle agitation, or any combination thereof.

Another example is disclosed in a United States Publication No. 20180264875, entitled “Cosmetic Brush Cleaner and Dryer” (“the '875 Publication”). The '875 Publication discloses a method and system for cosmetic brush cleaning and drying. It solves all the problems mentioned for the other products in the industry. The system cleans multiple brushes effectively at one time without user intervention. After cleaning, the device can be placed into a drying mode that dries the brushes many times faster than air-drying along. Also, a mechanism can be included that transitions the device from the cleaning mode to the drying mode without user intervention, thereby, creating a fully automated system. In addition, the system cleans brushes in only one minute. The system has four sub-assemblies/sub-systems/main components: Retractable power cord; Handle (which holds the makeup brushes during cleaning and drying); Base (which holds the cleaning solution); and Cradle (which fits between the base and the handle).

Another example is disclosed in U.S. Pat. No. 10,918,201, entitled “Brush cleaning device using UV light to dry, sanitize, and disinfect cosmetic brushes” (“the '201 Patent”). The '201 Patent discloses a dryer, sanitizer, and disinfecting device for make-up brushes includes an outer housing having an upper surface defining a plurality of openings therein with the plurality of openings arranged in a linear array; an inner housing including a retainer having a plurality of recesses corresponding to the plurality of openings defined in the outer housing; and a sterilizing component supported within at least one of the outer housing or the inner housing, wherein the sterilizing component effects the cleaning chamber, and wherein the sterilizing component includes a UV light bulb.

Yet another example is disclosed in a United States Publication No. 20060175554, entitled “Germicidal brush cleaner” (“the '554 Publication”). The '554 Publication discloses a germicidal brush cleaner that uses a germicidal light source to disinfect the individual bristles on a plurality of toothbrushes and a hairbrush. Each toothbrush and hairbrush includes bristles made of optical fibers capable of transmitting ultraviolet light. There is also a special method of attachment of the toothbrush inside the cleaner that secures the toothbrushes in a set position in the holder. The germicidal light source may be a germicidal fluorescent ultraviolet lamp. The light rays from the germicidal light source are directed at the opposite ends of the bristle at the critical angle, or slightly greater than the critical angle, in order to attain total internal reflection of the light down the bristles of the toothbrushes.

Although the above disclosures are useful, still there is a need for an improved cosmetic brush cleaning device for cleaning and sanitizing cosmetic brushes.

### SUMMARY

It is one of the main objects of the present invention to provide a cosmetic brush cleaning device and that avoids the drawbacks of the prior art.

It is another object of the present invention to provide a cosmetic brush cleaning device for cleaning and sanitizing cosmetic brushes.

It is another object of the present invention to provide a cosmetic brush cleaning device capable of holding a plurality of cosmetic brushes along with sanitization of the cosmetic brushes.

In order to overcome the limitations here stated, the present invention provides a cosmetic brush cleaning device for cleaning and sanitizing cosmetic brushes. The cosmetic brush cleaning device includes a first housing and a second housing placed over the first housing. The first housing includes a container having a first base, and first walls extending from the first base. The first walls receive brush holders. Further, the first housing includes a first door for providing and preventing access to the container. The second housing includes a second base, and second walls extending from the second base. The second base and the second walls form a brush receiving area. The second housing includes positioned on the second walls. The first housing includes a control panel positioned adjacent to the container. The first housing includes a fan, an Ultraviolet (UV) light, and a controller. The brush holders receive cosmetic brushes. The controller operates the fan and the UV light to clean and sanitize the cosmetic brushes. The cosmetic brushes are removed from the first housing and placed in the second housing to dry the cosmetic brushes.

In one aspect of the present invention, the container includes one or more meshes placed underneath the brush holders. The one or more meshes collect debris falling from the cosmetic brushes in the container. Each of the brush holders includes an elongated rod, and a connecting member connecting the elongated rod. The connecting member includes a hook and loop for receiving a cosmetic brush of the cosmetic brushes.

The first housing further includes a chamber. The chamber positions above the first housing. The chamber includes a rectangular compartment having an opening for receiving a palette. The rectangular compartment includes a sliding door for providing and restricting access to the opening. The fan and the UV light operate to clean and sanitize the palette.

The control panel includes a display for displaying information corresponding to operation of the said fan and the light. Further, the control panel includes a timer. The timer operates the fan and the light for a predetermined time period.

In one advantageous feature of the present invention, the cosmetic brush cleaning device eliminates 99.9% of airborne pollutants, such as bacteria and allergens, from being redeposited on the cosmetic brushes and removes unpleasant smells and odors from the cosmetic brushes.

In another advantageous feature of the present invention, the cosmetic brush cleaning device holds a complete brush set with pallet and blender storage. The second housing helps to store all types of brushes, sponges and the like and acts as a drying rack.

In another advantageous feature of the present invention, customization and branding are available. The design shall allow custom inserts, on all sides of the device excluding the control panel, displaying an artist's business logo or other designs to personalize or promote a brand.

Features and advantages of the invention hereof will become more apparent in light of the following detailed description of selected embodiments, as illustrated in the accompanying FIGUREs. As will be realized, the invention disclosed is capable of modifications in various respects, all without departing from the scope of the invention. Accordingly, the drawings and the description are to be regarded as illustrative in nature.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 illustrates an environment in which a cosmetic brush cleaning device implements, in accordance with one embodiment of the present invention;

FIG. 2 illustrates a perspective view of the cosmetic brush cleaning device, in accordance with one embodiment of the present invention;

FIGS. 3, 4 and 5 illustrate a first housing of the cosmetic brush cleaning device, in accordance with one embodiment of the present invention;

FIG. 6 illustrates a brush holder, in accordance with one embodiment of the present invention;

FIG. 7 illustrates a container, in accordance with one embodiment of the present invention;

FIG. 8 illustrates a second housing positioned over the first housing, in accordance with one embodiment of the present invention;

FIG. 9 illustrates a control panel in the first housing, in accordance with another embodiment of the present invention;

FIGS. 10A and 10B illustrate a top view and a bottom view, respectively of a top plate, in accordance with another embodiment of the present invention;

FIG. 11 illustrates brushes of varied sizes, in accordance with another embodiment of the present invention; and

FIG. 12 illustrates a block diagram of the control panel, in accordance with another embodiment of the present invention.

### DESCRIPTION OF THE EMBODIMENTS OF THE INVENTION

The following detailed description set forth below in connection with the appended drawings is intended as a description of exemplary embodiments in which the presently disclosed invention may be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed cosmetic brush cleaning device. However, it will be apparent to those skilled in the art that the presently disclosed invention may be practiced without these specific details. In some instances, well-known structures and devices are shown in functional or conceptual diagram form in order to avoid obscuring the concepts of the presently disclosed cosmetic brush cleaning device.

In the present specification, an embodiment showing a singular component should not be considered limiting. Rather, the invention preferably encompasses other embodiments including a plurality of the same component, and vice-versa, unless explicitly stated otherwise herein. Moreover, the applicant does not intend for any term in the specification to be ascribed an uncommon or special meaning unless explicitly set forth as such. Further, the present invention encompasses present and future known equivalents to the known components referred to herein by way of illustration.

Although the present invention provides a description of a device, it is to be further understood that numerous changes may arise in the details of the embodiments of the device. It is contemplated that all such changes and additional embodiments are within the spirit and true scope of this disclosure.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure.

The present invention discloses a cosmetic brush cleaning device for cleaning and sanitizing cosmetic brushes. The cosmetic brush cleaning device includes a first housing and a second housing placed over the first housing. The first housing includes a container having a first base, and first walls extending from the first base. The first walls receive brush holders. The second housing includes a second base, and second walls extending from the second base. The second base and the second walls form a brush receiving area. The first housing includes a control panel positioned adjacent to the container. The first housing includes a fan, an Ultraviolet (UV) light, and a controller. The brush holders receive cosmetic brushes. The controller operates the fan and the UV light to clean and sanitize the cosmetic brushes. The cosmetic brushes are removed from the first housing and placed in the second housing to dry the cosmetic brushes.

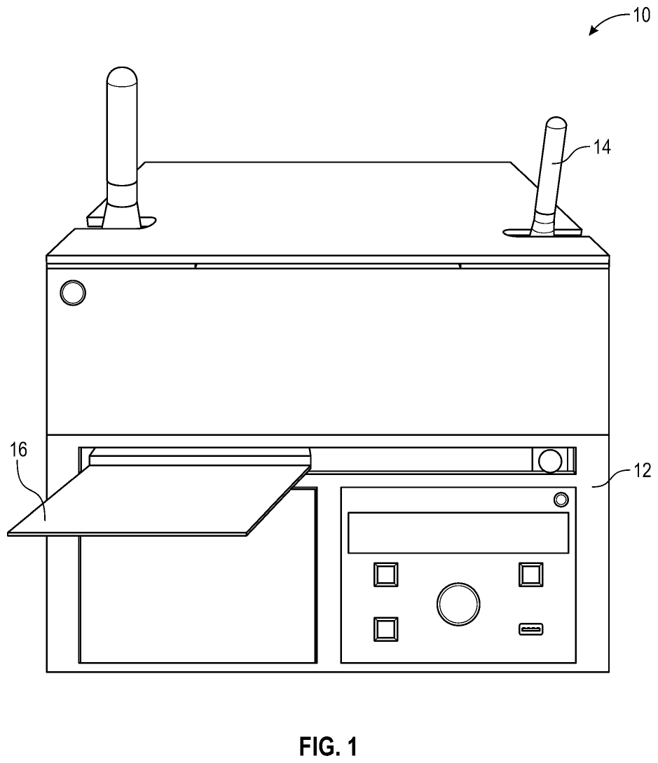

Various features and embodiments of a cosmetic brush cleaning device for cleaning and sanitizing cosmetic brushes are explained in conjunction with the description of FIGS. 1-11.

Referring to FIG. 1, an environment 10 in which a cosmetic brush cleaning device 12 implements is shown, in accordance with one exemplary embodiment of the present invention. Cosmetic brush cleaning device 12 helps to clean/sanitize or dry cosmetic brushes (or simply brushes) 14 and/or one or more palettes 16.

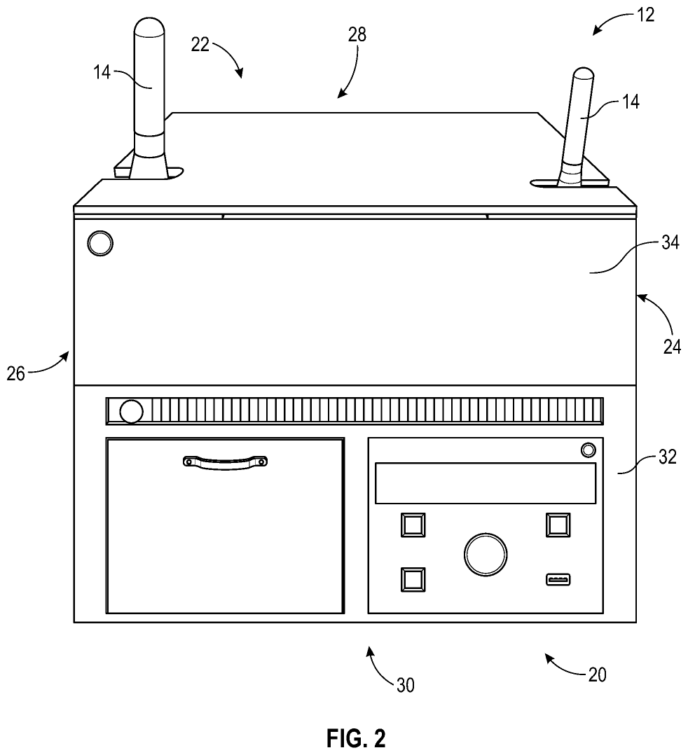

FIG. 2 shows a perspective view of cosmetic brush cleaning device 12, in accordance with one embodiment of the present invention. Cosmetic brush cleaning device 12 includes a front end 20, a rear end 22, a first side 24, a second side 26, a top end 28 and a bottom end 30. In accordance with the present invention, cosmetic brush cleaning device 12 includes a first housing 32 and a second housing 34. Here, first housing 32 indicates a bottom housing and second housing 34 indicates a top housing. As can be seen from at least FIG. 2, second housing 34 positions over first housing 32.

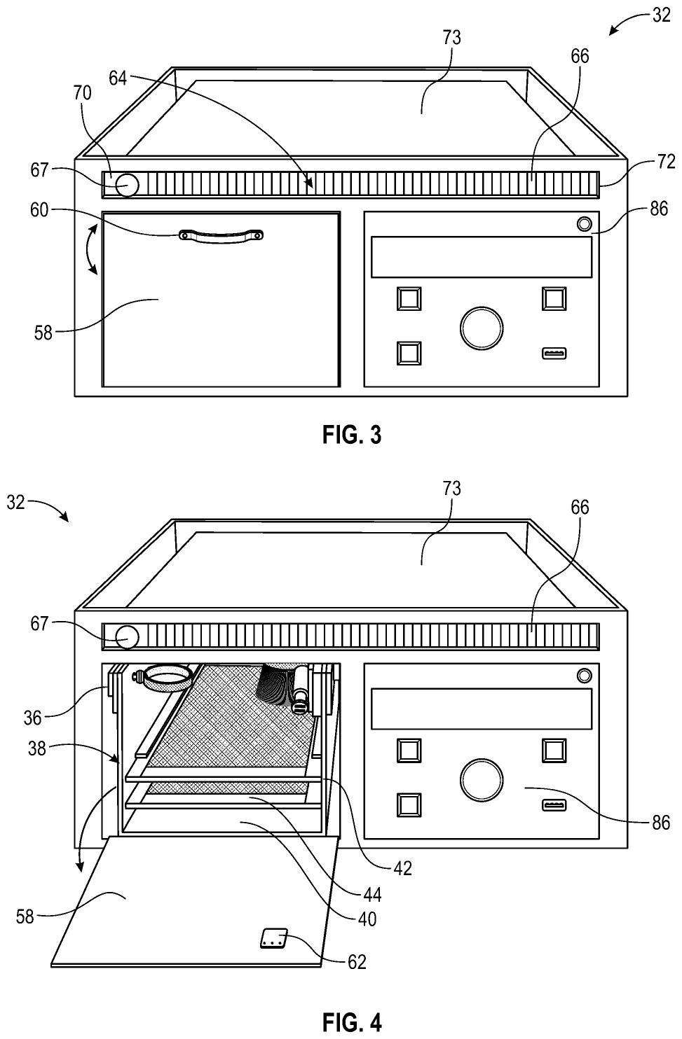

FIGS. 3, 4, and 5 show perspective views of first housing 32, in accordance with one embodiment of the present invention. First housing 32 is made of a metal, hard plastic or any other suitable material. In one example, bottom housing 32 comes in a rectangular or square box-like configuration. First housing 32 presents a first compartment 36. First compartment 36 indicates an open area or hollow area within first housing 32. In the present invention, first compartment 36 receives a container 38. Container 38 indicates a structure capable of holding meshes 44 and/or brushes 56 of various shapes and sizes in order to clean and/or sanitize brushes 14, 56.

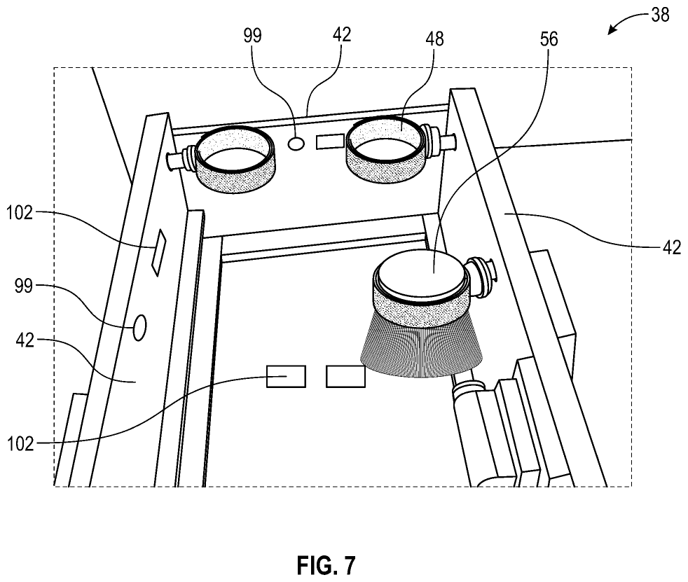

Container 38 includes a flat base 40 and walls 42 extending from three sides (rear end 22, first side 24 and second side 26). In one exemplary embodiment, walls 42 at first side 24 and second side 26 include a plurality of holes 46. Holes 46 position at different places over walls 42 and come in various sizes. Holes 46 help to removably connect brush holders 48 to walls 44. Further, FIG. 7 shows a top perspective view of container 38. In one example, base 40 and walls 44 include a plurality of lights 102. Lights 102 include Ultraviolet (UV) lights capable of producing electromagnetic energy in order to destroy microorganisms to reproduce and causes inactivation of microbes. Lights 102 position at inner side of base 40 and walls 44 and help to direct UV light onto brushes 56 placed in container 38. In one embodiment, walls 44 include blowers 99 for supplying air from fan 98.

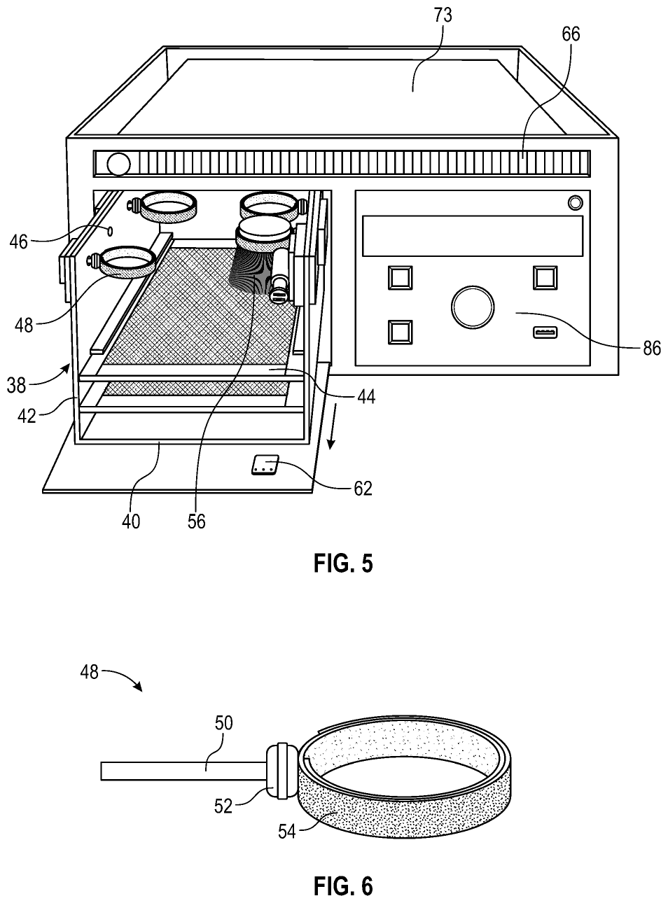

FIG. 6 shows a side perspective view of a brush holder 48, in accordance with one embodiment of the present invention. Brush holder 48 includes an elongated rod 50. Elongated rod 50 connects to a connecting member 52. Connecting member 52 has a slightly larger diameter than elongated rod 50 and aids in connecting brush holder 48 to wall 44. Further, connecting member 52 presents a strap 54. Strap 54 includes a hook and loop e.g., Velcro™. Strap 54 is adjusted to receive a brush 56 of different sizes. In the present invention, brush holders 46 help to hang or hold multiple brushes 56 upside down. In case brushes 56 are longer, then one or more meshes 44 are removed to place them vertically with support from brush holders 46.

Further, first housing 32 includes a door 58. Door 58 connects to base 40. In the present invention, door 58 swings open, as shown in FIG. 4. In one example, door 58 includes a handle 60 at front end 20, as shown in FIG. 3. A user (not shown) holds handle 60 and swings down door 58 to access container 38. In one example, door 58 includes a door connecting member 62. Door connecting member 62 indicates a magnet or latch-like member that allows connecting door 58 to container 38. In use, the user opens door 58 and pulls container 38 forward i.e., towards front end 20 to place or to remove brushes 56 in container 38, as shown in FIG. 5.

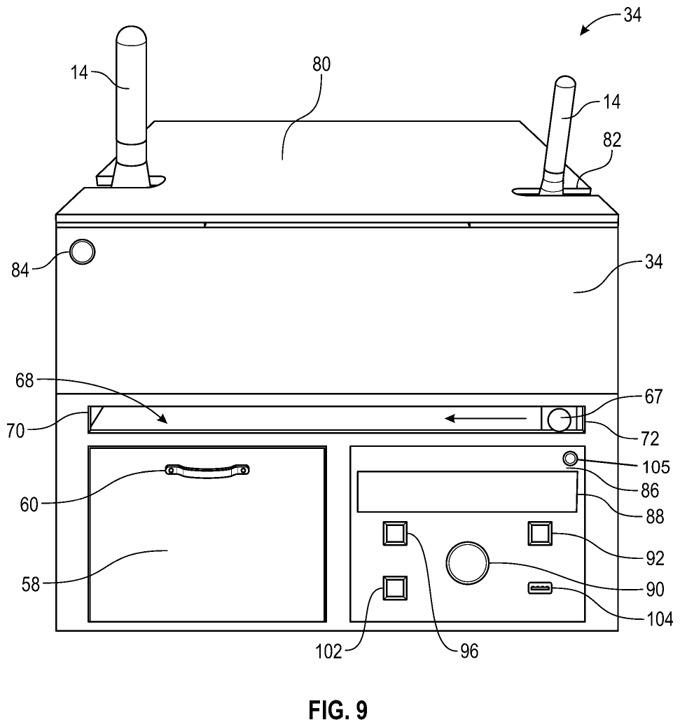

In the present invention, first housing 32 includes a chamber 64. Chamber 64 positions over container 38. Chamber 64 presents a rectangular compartment 73 extending at the interior of first housing 32. Chamber 64 includes a slider door 66 that is slidable with the help of a knob 67. Slider door 66 is operable from first end 70 to second end 72 of chamber 64. Upon sliding, sliding door 66 provides an opening 68 (as shown in FIG. 9) for placing one or more palettes 16 in rectangular compartment 73.

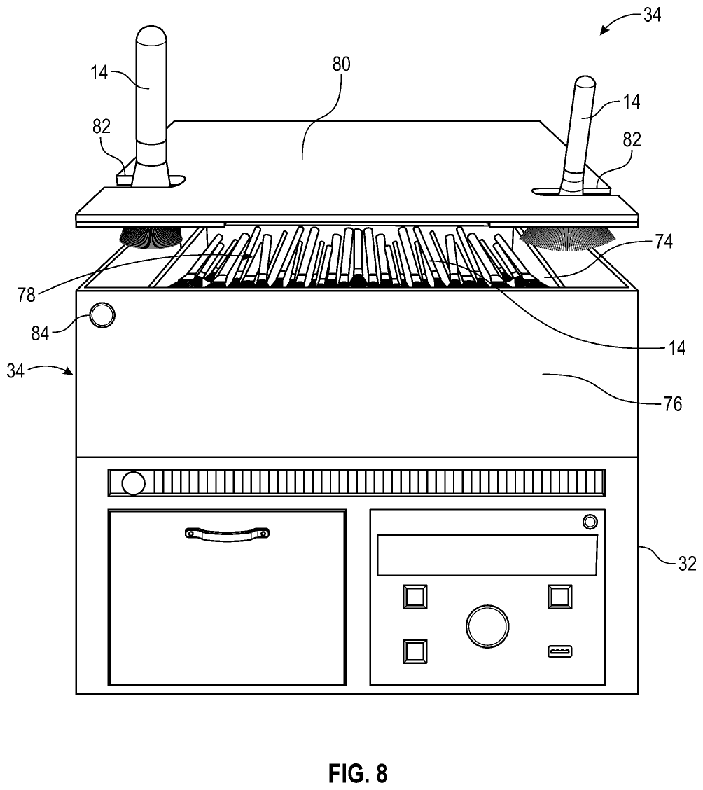

Referring to FIG. 2, cosmetic brush cleaning device 12 having second housing 34 positioned over first housing 32 is shown. Further, FIG. 8 shows the components of second housing 34. As can be seen, second housing 34 includes a flat base 74. Base 74 sits over first housing 32. Second housing 34 encompasses walls 76 extending perpendicularly from base 74. In one implementation, base 74 includes one or more holes (not shown) for receiving blowers to supply air to dry brushes 14 placed in second housing 34. Optionally, walls 76 are provided with UV lights 102 to sanitize brushes 14 placed in second housing 34. Base 74 and walls 76 form a brush receiving area 78. Brush receiving area 78 indicates a hollow area within second housing 34 for receiving brushes 14, 56 of various shapes and sizes. Further, second housing 34 includes a top plate 80. Top plate 80 rests over walls 76 and helps to retain brushes 14 within second housing 34. Top plate 80 encompasses one or more cut sections 82 at first side 24 and/or second side 26. Cut sections 82 receive brushes 14, 56 such that bristles of brushes 14 are made to go inside brush receiving area 78, as shown in FIG. 8. In one embodiment, second housing 34 encompasses an actuator 84. Actuator 84 positions at the outer side of walls 76. Actuator 84 operates top plate 80. For example, pressing actuator 84 lifts up top plate 80 to access brush receiving area 78 in order to place or remove brushes 14, 56 from second housing 34.

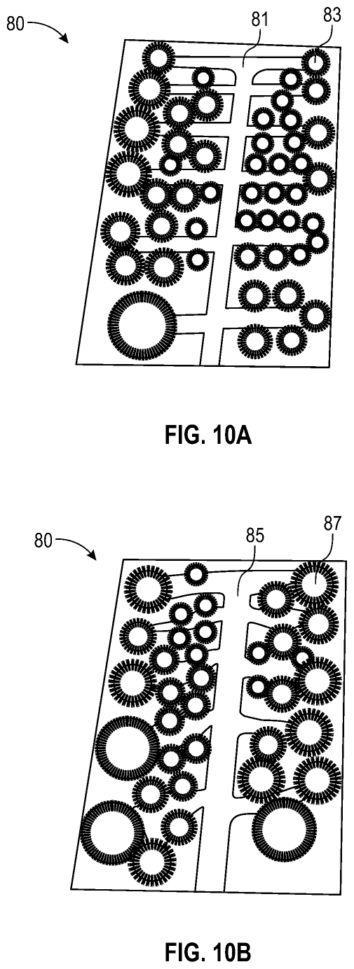

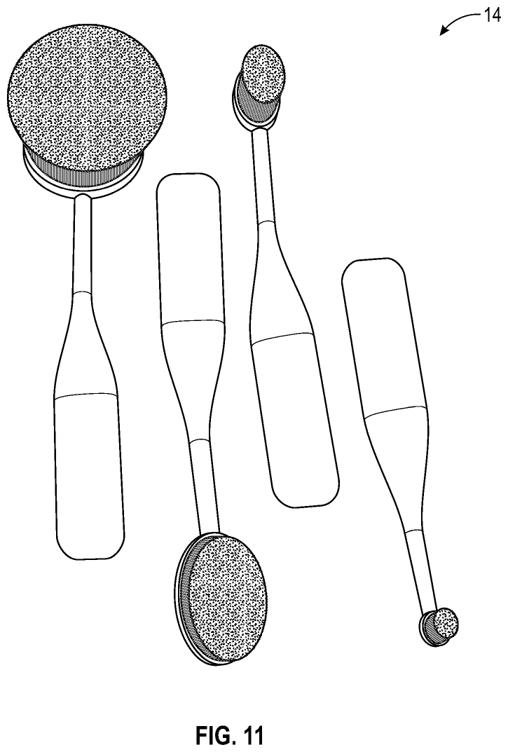

FIGS. 10A and 10B show a top view and a bottom view, respectively of top plate 80, in accordance with another embodiment of the present invention. On top side, top plate 80 first cut sections 81 having first brush receiving portions 83. Brush receiving portions 83 receive brushes 14 of varied sizes, as shown in FIG. 11. In use, a user flips top plate 80 such that the other side of top plate 80 is made to face up as shown in FIG. 10B, for example. Here, top plate 80 include second cut sections 85 having second brush receiving portions 87. In one example, first brush receiving portions 83 are more in number than second brush receiving portions 87. For instance, first brush receiving portions 83 receive 49 brushes while second brush receiving portions 87 receive 36 brushes. In another example, first brush receiving portions 83 are smaller in size than second brush receiving portions 87. In use, the user flips top plate and places brushes 14 depending on their size to sanitize brushes.

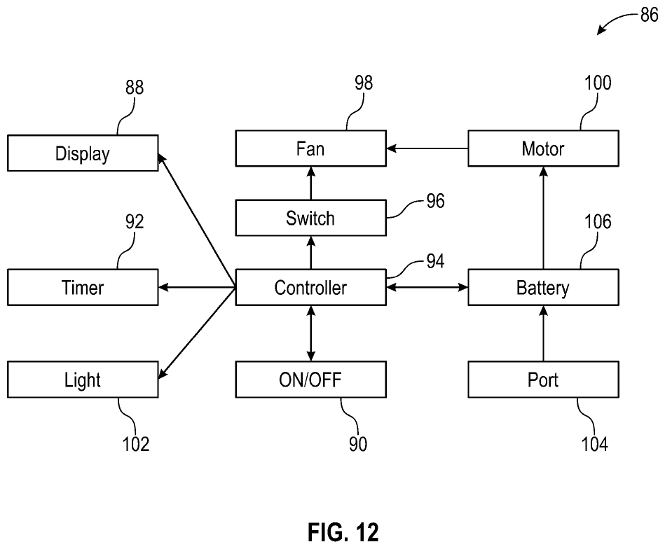

Further, first housing 32 includes a control panel 86 positioned adjacent to first compartment 36, as shown in FIG. 9. Further, FIG. 12 shows a block diagram of control panel 86, in accordance with one embodiment of the present invention. Control panel 86 includes a time/date display 88, an ON/OFF button 90, a timer 92, a Bluetooth connection indicator 105, and a controller 94. Time/date display 88 displays data/information corresponding to the operation of cosmetic brush cleaning device 12. ON/OFF button 90 helps to start or stop the operation of cosmetic brush cleaning device 12. Timer 92 helps to set a time of duration for operating cosmetic brush cleaning device 12. Controller 94 indicates a processor or microcontroller configured to process the signals and/or data received by the components in cosmetic brush cleaning device 12.

Control panel 86 further includes a manual switch 96 for operating a fan 98. In one implementation, fan 98 is positioned within one of first housing 32 and second housing 34. Fan 98 supplies air into first housing 32 and second housing 34 in order to clean and sanitize brushes 14, 56. In one example, fan 98 connects to a motor 100. Motor 100 operates using power drawn from a battery 106. In one example, battery 106 gets charged via a charging port 104. Further, control panel 86 includes a light 102. Light 102 includes an ultraviolet light capable of providing required UV radiation to purify and/or disinfect air.

In use, the user opens door 58, slides out container 38 and places one or more brushes 14, 56 at brush holders 48. Upon placing, the user pushes container 38 into first compartment 36 and closes door 58. If needed, the user places palette 16 in chamber 64 and adjusts slider door 66. Subsequently, the user operates the ON/OFF button 90 to initiate the operation to clean and sanitize brushes 14, 56 and palette 16. In one example, the user determines a predetermined time period to operate fan 98 and light 102 to clean and disinfect brushes 14, 56 and palette 16. Here, controller 94 operates fan 98 and light 102 for the predetermined time period. In order to disinfect/sanitize, light 102 emits UV radiation and destroys the nucleic acid and disrupts the DNA of germs on brushes 14, 56 and palette 16. Further, fan 98 blows air such that excess dust or debris remaining in brushes 14, 56 and palette 16 are made to fall over mesh 44. After completion of the predetermined time period, controller 94 instructs fan 98 and light 102 to turn OFF.

Further, the user opens door 58 and removes brushes 14, 56 and palette 1. Subsequently, the user places brushes 14, 56 and/or palette 16 in second housing 34 i.e., in brush receiving area 78. Optionally, the user places brushes 14, 56 at cut portions 82 for drying places brushes 14, 56. From the above, a person skilled in the art understands first housing 32 is used to clean and sanitize brushes 14, 56 and/or palette 16. Further, second housing 34 is used as a drying rack for drying brushes 14, 56 and/or palette 16 after cleaning.

A person skilled in the art appreciates that the cosmetic brush cleaning device may come in a variety of shapes and sizes depending on the need and comfort of the user. Further, many changes in the design and placement of components may take place without deviating from the scope of the presently disclosed cosmetic brush cleaning device.

In the above description, numerous specific details are set forth such as examples of some embodiments, specific components, devices, methods, in order to provide a thorough understanding of embodiments of the present invention. It will be apparent to a person of ordinary skill in the art that these specific details need not be employed, and should not be construed to limit the scope of the invention.

In the development of any actual implementation, numerous implementation-specific decisions must be made to achieve the developer's specific goals, such as compliance with system-related and business-related constraints. Such a development effort might be complex and time-consuming, but may nevertheless be a routine undertaking of design, fabrication, and manufacture for those of ordinary skill. Hence as various changes could be made in the above constructions without departing from the scope of the invention, it is intended that all matter contained in the above description or shown in the accompanying drawings shall be interpreted as illustrative and not in a limiting sense.

The foregoing description of embodiments is provided to enable any person skilled in the art to make and use the invention. Various modifications to these embodiments will be readily apparent to those skilled in the art, and the novel principles and invention disclosed herein may be applied to other embodiments without the use of the innovative faculty. It is contemplated that additional embodiments are within the spirit and true scope of the disclosed invention.

What is claimed is:

1. A cosmetic brush cleaning device for cleaning and sanitizing cosmetic brushes, said cosmetic brush cleaning device comprising:
a first housing, said first housing comprising:
a container comprising:
a first base;
first walls extending from said first base,
wherein said first walls comprise a plurality of holes; and
brush holders removably connected to said plurality of holes;
a second housing, wherein said second housing positions above said first housing, wherein said second housing comprising:
a second base;
second walls extending from said second base, wherein said second base and said second walls form a brush receiving area; and
a top plate, wherein said top plate positions over said second walls and helps to close said second housing with said second walls; and
a control panel, wherein said control panel positions adjacent to said container in said first housing, wherein said control panel comprising:
a fan;
an Ultraviolet (UV) light; and
a controller,
wherein said brush holders receive cosmetic brushes, wherein said controller operates said fan and said UV light to clean and sanitize said cosmetic brushes in said first housing, and
wherein said cosmetic brushes are removed from said first housing and placed in said second housing to dry said cosmetic brushes.

2. The cosmetic brush cleaning device of claim 1, wherein said container comprises one or more meshes placed underneath said brush holders, and wherein said one or more meshes collect debris falling from said cosmetic brushes.

3. The cosmetic brush cleaning device of claim 2, wherein each of said brush holders comprises an elongated rod, and a connecting member connecting said elongated rod, and wherein said connecting member comprises a hook and loop.

4. The cosmetic brush cleaning device of claim 3, wherein said hook and loop receives a cosmetic brush of said cosmetic brushes.

5. The cosmetic brush cleaning device of claim 1, wherein said first housing comprises a first door for providing and preventing access to said container.

6. The cosmetic brush cleaning device of claim 1, wherein said first housing further comprises a chamber, wherein said chamber positions above said first housing, wherein said chamber comprises a rectangular compartment having an opening for receiving a palette, and wherein said fan and said UV light operate to clean and sanitize said palette.

7. The cosmetic brush cleaning device of claim 6, wherein said rectangular compartment comprises a sliding door for providing and restricting access to the opening.

8. The cosmetic brush cleaning device of claim 1, wherein said top plate comprises one or more cut sections, and wherein said one or more cut sections receive said cosmetic bushes facing said brush receiving area to dry said cosmetic brushes.

9. The cosmetic brush cleaning device of claim 1, wherein said control panel comprises a display for displaying information corresponding to operation of the said fan and said light.

10. The cosmetic brush cleaning device of claim 1, wherein said control panel comprises a timer, and wherein said timer operates said fan and said light for a predetermined time period.

11. A method of providing a cosmetic brush cleaning device for cleaning and sanitizing cosmetic brushes, said method comprising the steps of:
providing a first housing comprising a container having a first base and first walls extending from said first base, said first walls comprising a plurality of holes;
providing brush holders removably connected to said plurality of holes;
providing a second housing positioned above said first housing, said second housing comprising a second base, and second walls extending from said second base, said second base and said second walls forming a brush receiving area;
providing a top plate positioned over said second walls to close said second housing;
providing a control panel adjacent to said container in said first housing, said control panel comprising a fan, an Ultraviolet (UV) light, and a controller;
receiving cosmetic brushes at said brush holders;
operating said fan and said UV light via said controller to clean and sanitize said cosmetic brushes; and
removing said cosmetic brushes from said first housing and placing in said second housing to dry said cosmetic brushes.

12. The method of claim 11, further comprising providing one or more meshes underneath said brush holders for collecting debris falling from said cosmetic brushes.

13. The method of claim 12, further comprising providing a hook and loop in each of said brush holders.

14. The method of claim 13, further comprising receiving a cosmetic brush of said cosmetic brushes in said hook and loop.

15. The method of claim 11, further comprising providing a first door in said first housing for providing and preventing access to said container.

16. The method of claim 11, further comprising:
providing a chamber, said chamber positioning above said first housing;
providing a rectangular compartment in said chamber, said rectangular compartment having an opening for receiving a palette; and
operating said fan and said UV light to clean and sanitize said palette.

17. The method of claim 16, further comprising providing a sliding door in said rectangular compartment for providing and restricting access to the opening.

18. The method of claim 11, further comprising providing one or more cut sections at said top plate, said one or more cut sections receiving said cosmetic bushes facing said brush receiving area to dry said cosmetic brushes.

19. The method of claim 11, further comprising providing a display in said control panel for displaying information corresponding to operation of the said fan and said light.

20. The method of claim 11, further comprising providing a timer in said control panel, said timer operating said fan and said light for a predetermined time period.
